(19) United States
(12) Patent Application Publication
(10) Pub. No.: US 2014/0242019 A1
(43) Pub. Date: Aug. 28, 2014

(54) COMPOSITION FOR PREVENTING HAIR LOSS AND GROWING HAIR COMPRISING HYDROXYDIHYDROBOVOLIDE

(71) Applicant: Pukyong National University Industry-University Cooperation Foundation, Busan (KR)

(72) Inventors: Se-Kwon Kim, Busan (KR); Soon-Sun Bak, Busan (KR); Sang-Man Lee, Busan (KR)

(73) Assignee: Pukyong National University Industry-University Cooperation Foundation, Busan (KR)

(21) Appl. No.: 14/108,266

(22) Filed: Dec. 16, 2013

(30) Foreign Application Priority Data
Feb. 26, 2013 (KR) 10-2013-0020376

(51) Int. Cl. A61K 8/49; A61Q 7/00; A61K 8/97

(52) U.S. Cl. CPC A61K 8/4973; A61K 8/97; A61Q 7/00
USPC 424/74; 424/70.1

(57) ABSTRACT

There is provided a new use of a hydroxydihydrobovolide compound having effects on preventing hair loss and growing hairs. Since the hydroxydihydrobovolide compound has effects on increasing survival rates of human hair follicle dermal papilla cells and hair outer root sheath cells and also growing a length of hairs, the compound is of great value as a pharmaceutical composition or cosmetic composition for improving scalp or preventing hair loss. Further, since the hydroxydihydrobovolide compound is isolated and purified from Eucheuma cottonii that is red algae and does not induce cytotoxicity, it is stable in the body and thus can be used as a raw material for functional health food.

### CROSS-REFERENCE TO RELATED APPLICATIONS

This application claims the priority of Korean Patent Application No. 2013-0020376 filed on Feb. 26, 2013, in the Korean Intellectual Property Office, the disclosure of which is incorporated herein by reference.

### BACKGROUND OF THE INVENTION

1. Field of the Invention

The present invention relates to a new use of hydroxydihydrobovolide compound that is effective in preventing hair loss and growing hairs.

2. Description of the Related Art

Recently, along with an explosion of interest in appearance image, hair loss is one of the biggest worry. In general, the hair loss has viewed only as male problem in their 40s to 50s. However, recently 20s to 30s patients with hair loss are skyrocketing and the number of female patients with hair loss is increasing. Such hair loss is increasing due to many environment factors such as the imbalance of dietary life of modern day people, harmful air, tainted drinking water, acid rain, and stress. For this reason, a number of related studies on preventing and treating hair loss are currently in progress.

Hair loss means the state where there are no hairs in the area to be normally needed to have hairs or the phenomenon where hairs stopped growing naturally fall off. In general, about 50 to 100 or more of hairs are coming off in a day. It is called alopecia that the number of hairs that fall off increases. There are a great variety of causes of hair loss. It is understood that bald head occurs due to genetic causation and androgen that is a male hormone as an important factor. In the case of female hair loss, it is estimated that it partly occurs due to the same route as male hair lose. It is understood that pattern baldness is an autoimmune disease. In addition, telogen alopecia occurs due to physical and mental severe stress such as endocrinopathy, nutrient deficiency, drug use, childbirth, fever, and surgery. In the case of telogen alopecia, a portion of hairs fulfill their growth period and then be in the state of telogen, resulting in occurring hair loss as temporary hair loss.

In order to prevent and treat such hair loss, various attempts have been done, and there are surgery manners, such as scalping flap, reduction of hair loss area, tissue expander, and follicular unit hair transplantation, and also drugs for preventing hair loss and growing hairs using oriental medicine extracts such as a natural substance are used. Such a surgery manner has superior potency in a short period time, but economic and mental burdens are present as the biggest problem. For this reason, hair loss is being prevented and avoided through various external applications instead of the surgery manner. However, in general, in the case of using a synthetic preparation, there could be side effects. Examples of the side effects may include loss of sex function such as hyposexuality and erectile dysfunction, and also there is a risk leading to fatal deformities in the case of female.

Accordingly, a composition for preventing hair loss and growing hairs, in which the composition has excellent therapy effect without side effects, is needed.

### SUMMARY OF THE INVENTION

Accordingly, the present inventors investigated that a hydroxydihydrobovolide compound isolated from an extract of Eucheuma cottonii that is a kind of red algae has an activity capable of preventing hair loss and growing hairs. Thus, the inventors completed the present invention.

Thus, an object of the present invention is to provide a pharmaceutical composition for preventing hair loss and growing hairs, in which the composition includes hydroxydihydrobovolide as an effective component.

Further, another object of the present invention is to provide health functional food for preventing hair loss and growing hairs, in which the composition includes hydroxydihydrobovolide as an effective component.

Furthermore, still another object of the present invention is to provide a cosmetic composition for preventing hair loss and growing hairs, in which the composition includes hydroxydihydrobovolide as an effective component.

In order to achieve the above-mentioned objects, the present invention provides a pharmaceutical composition for preventing hair loss and growing hairs, in which the composition includes hydroxydihydrobovolide represented by the following Chemical Formula 1 as an effective component.

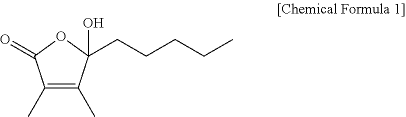

According to an embodiment of the present invention, the hydroxydihydrobovolide may be isolated from an extract of Eucheuma cottonii that is red algae.

According to an embodiment of the present invention, the hydroxydihydrobovolide may increase survival rates of human hair follicle dermal papilla cells and/or hair outer root sheath cells.

According to an embodiment of the present invention, the hydroxydihydrobovolide may have an activity capable of growing a length of human hairs.

According to an embodiment of the present invention, the extract may be extracted by using water or an organic solvent.

According to an embodiment of the present invention, the extract may include an extract, a concentrate, or a dry matter of Eucheuma cottonii.

According to an embodiment of the present invention, the concentration of the hydroxydihydrobovolide may be 0.005 to 10 μM.

According to an embodiment of the present invention, the pharmaceutical composition may be a formulation selected from the group consisting of creams, gels, patches, spraying agents, ointments, a hardening agent, lotions, liniments, pastas, and cataplamas as a skin external application.

In addition, the present invention provides health functional food for preventing hair loss and growing hairs, in which the composition includes hydroxydihydrobovolide represented by the following Chemical Formula 1 as an effective component.

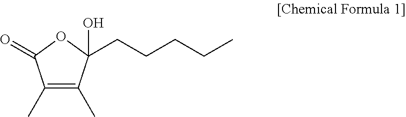

Furthermore, the present invention provides a cosmetic composition for preventing hair loss and growing hairs, in which the composition includes hydroxydihydrobovolide represented by the following Chemical Formula 1 as an effective component.

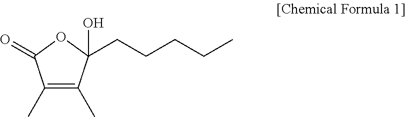

According to an embodiment of the present invention, the cosmetic composition may be any one formulation selected from the group consisting of hair tonics, nutritional cosmetic preparations for hair care, scalp treatments, hair treatments, hair rinses, hair shampoos, and hair lotions.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 shows a structure of the hydroxydihydrobovolide compound according to the present invention;

FIG. 2 shows a result of analyzing a methanol extract of Eucheuma cottonii by using a HPLC;

FIG. 3 shows a result of observing an effect of the hydroxydihydrobovolide compound according to the present invention on cell proliferation by using human hair follicle dermal papilla cells and hair outer root sheath cells; and

FIG. 4 shows a result of measuring an effect of the hydroxydihydrobovolide compound according to the present invention on hair growth by using a human hair tissue culturing method.

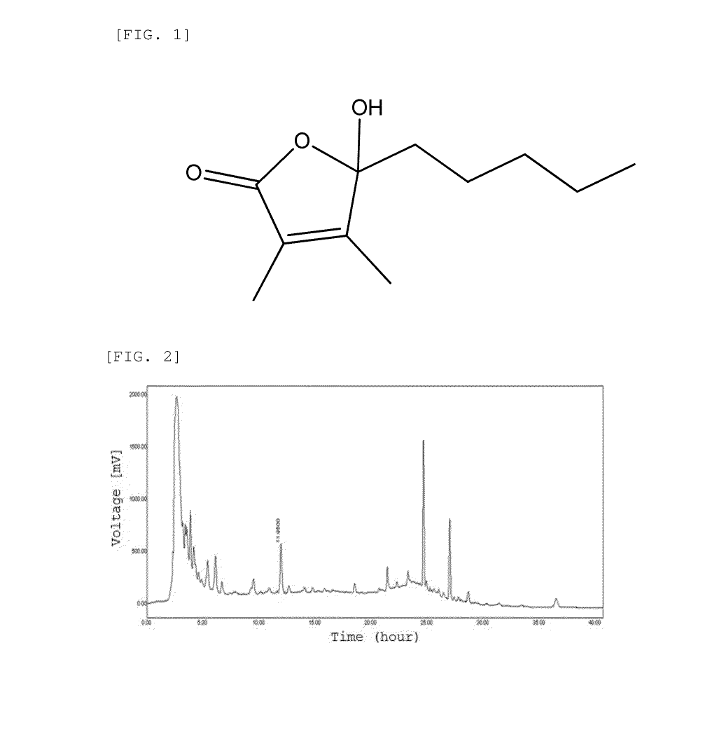

### DETAILED DESCRIPTION OF THE PREFERRED EMBODIMENT

Exemplary embodiments of the present invention will now be described in detail with reference to the accompanying drawings.

The present invention provides a new use of a hydroxydihydrobovolide compound as a new material for preventing hair loss and growing hairs.

Specifically, the hydroxydihydrobovolide compound according to the present invention is isolated from Eucheuma cottonii that is red algae and has a chemical name, 5-hydroxy-3,4-dimethyl-5-pentylfuran-2(5H)-one represented by the following Chemical Formula.

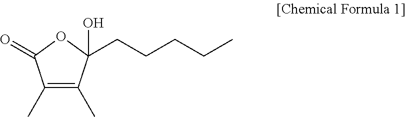

The hydroxydihydrobovolide compound according to the present invention may naturally be isolated or may also be prepared by using a chemical synthesis method that is known in the related art. Preferably, it may be isolated and purified from a solvent extract obtained from Eucheuma cottonii.

Further, in this time, any of Eucheuma cottoniis is available as long as it is commercially available from the market. Preferably, it needs to be sufficiently dried before being extracted by using a solvent. As a preferable specific example, the dried Eucheuma cottonii is reflux-extracted by using alcohol, and preferably, methanol, and the number of extraction is 1 to 5 times, and preferably 3 times.

In order to isolate a main active component of the above faction, peaks designated by using a reverse phase column (Triart C-18, YMC) and a HPLC were collected and then concentrated. In order to identify a material structure, the hydroxydihydrobovolide according to the present invention was isolated and purified by performing a proton-NMR, a carbon-NMR, and COSY, DEPT, HMBC, and HMQC.

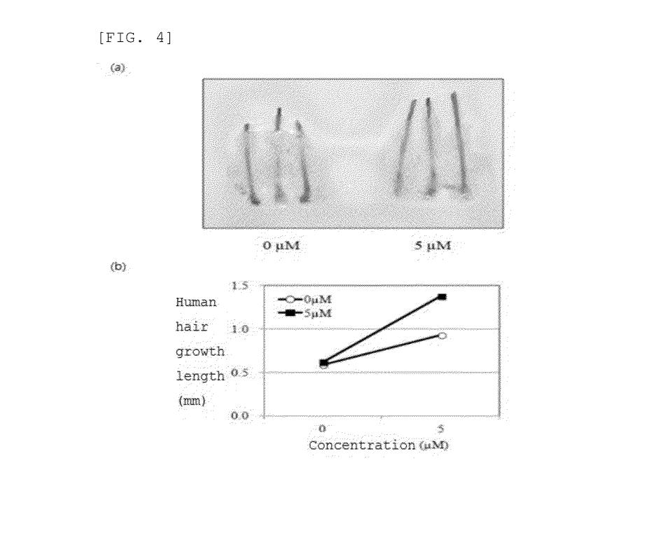

Accordingly, the present inventors confirmed that the hydroxydihydrobovolide compound isolated from Eucheuma cottonii has excellent effect on growing a length of human hairs, and thus can be used as a composition for preventing hair loss and growing hairs. According to an embodiment of the present invention, it can be confirmed that when the hydroxydihydrobovolide compound of the present invention is treated in the amount of 0.005 to 0.5 μM to human hair follicle dermal papilla cells, the compound shows a significant cell proliferation effect as compared with a control group without any treatment. Especially, in the case of treating the compound in the concentration of 0.05 μM, it is confirmed that the cell proliferation is increased by 144%. Furthermore, it can be confirmed that when the same test material is treated to hair outer root sheath cells, in the case of the treatment concentration of 0.005 to 0.5 μM, the material shows significantly higher cell proliferation rate than the control group, and especially, in the case of the treatment concentration of 0.5 μM, the material shows a cell proliferation increasing rate of 156%, the highest value, as compared with the control group (see FIGS. 3a and 3b).

According to another embodiment of the present invention, in order to confirm whether or not the hydroxydihydrobovolide compound of the present invention shows a hair growth effect, the hydroxydihydrobovolide compound in the concentration of 5 μM and the control group are compared. As a result, it can be confirmed that in the case of the group that is treated with the compound of the present invention, the human hair length is increased and also increased by 1.48 times or more longer than that of the control group at 6 days of the experiment (see FIG. 4).

Accordingly, the hydroxydihydrobovolide compound according to the present invention may be used as a new material for preventing hair loss and growing hairs.

The composition for preventing hair loss and growing hairs that is provided in the present invention may include a hydroxydihydrobovolide compound as an effective component. Further, the compound according to the present invention may include an extract, a concentrate, or a dry matter of Eucheuma cottonii.

In addition, the extract of Eucheuma cottonii may be an extract that is prepared by adding an organic solvent to Eucheuma cottonii that is red algae and then extracting. The extract may be obtained by using a solvent selected from water including purified water, lower alcohol having 1 to 4 carbon atoms such as methanol, ethanol, and butanol, or mixed solvent thereof, and preferably the mixed solvent of water and methanol, and may be obtained by using preferably 50 to 100% methanol and more preferably 70% methanol.

The composition including the hydroxydihydrobovolide compound of the present invention as an effective component may be used for a pharmaceutical composition and cosmetic composition for preventing hair loss and growing hairs.

In addition, the hydroxydihydrobovolide compound according to the present invention may be used in a type of salt, preferably, a pharmaceutically acceptable salt. The salt may be preferably acid addition salts formed by pharmaceutically acceptable free acid, and the free acid may be organic acid and inorganic acid. Examples of the organic acid may include, but are not limited to, citric acid, acetic acid, lactic acid, tartaric acid, maleic acid, fumaric acid, formic acid, propionic acid, oxalic acid, trifluoroacetic acid, benzoic acid, gluconic acid, methasulfonic acid, glycolic acid, succinic acid, 4-toluenesulfonic acid, glutamic acid, and aspartic acid. Furthermore, examples of the inorganic acid may include, but are not limited to, hydrochloric acid, bromic acid, sulfuric acid, and phosphoric acid.

Further, the composition according to the present invention may include singly a pharmaceutically effective amount of the hydroxydihydrobovolide compound or in combination of at least one of a pharmaceutically acceptable carrier, excipient, or diluents. The pharmaceutically effective amount means a sufficient amount to induce prevention of hair loss or growth of hair. The hydroxydihydrobovolide compound may be included in a concentration of 0.005 to 5 μM relative to the composition or 0.01 to 70% by weight relative to the total weight of the composition.

Further, the pharmaceutically effective amount of the hydroxydihydrobovolide compound according to the present invention is 0.5 to 100 mg/day/weight (kg) and preferably 0.5 to 10 mg/day/weight (kg). However, the pharmaceutically effective amount may appropriately be changed according to a degree of hair loss symptom, an age, a body weight, a health condition, sex, an administration route, a treatment period of a patient, and the like.

Further, the term “pharmaceutically acceptable composition” means a composition that is physiologically acceptable and does not generally cause an allergic reaction such as gastroenteric trouble and dizziness or a similar response thereof. Examples of the carrier, excipient and diluents may include lactose, dextrose, sucrose, solbitol, mannitol, xylitol, erythritol, maltitol, starch, acacia rubber, alginate, gelatin, calcium phosphate, calcium silicate, cellulose, methyl cellulose, polyvinyl pyrrolidone, water, methylhydroxybenzoate, propylhydroxybenzoate, talc, magnesium stearate, and mineral oils. In addition, filler, anticoagulant, a lubricant, a wetting agent, flavoring, an emulsifying agent, preservative, and the like may be further included.

In addition, the composition of the present invention may be formulated by using a method that is known in the related art so as to provide prompt release, continuous release, and delayed release of active components after being administrated to a mammal. The formulation type may be a type of powders, granules, tablets, an emulsion, syrups, aerosols, soft or hard gelatin capsules, sterilized injection solutions, or sterilized powders.

The composition according to the present invention may be administrated through various routes including an oral, a percutaneous injection, a subcutaneous injection, an intravenous injection or a muscle injection. The administration dose of the active component may appropriately be selected according to various factors, such as an administration route, an age, a sex, a body weight, and severity of a patient. In addition, the composition for preventing hair loss and growing hairs of the present invention may be used by mixing with a hair tonic or hair growth promoter that is conventionally used in the range, in which the function for preventing hair loss is not impaired.

Accordingly, the present invention may provide a pharmaceutical composition or cosmetic composition for preventing hair loss and growing hair, in which the composition includes a composition including a hydroxydihydrobovolide compound.

Further, since the hydroxydihydrobovolide compound according to the present invention does not induce cytotoxicity and does not cause a side effect, it is stable in the body and thus can be used to the body in relief, and thereby it can be used as a composition for food to prevent hair loss and grow hairs.

A food composition for preventing hair loss and growing hairs, in which the composition includes the hydroxydihydrobovolide compound as an effective component, may be easily used as food having an effect on growing hairs, such as a main material of food, a supplement material of food, foods supplements, functional foods, or drinks.

In the present specification, the term “food” means a natural substance or processed goods including one or more nutrients, and preferably, one that is in the state of being edible through some processing processes. As a typical meaning, it means one including all of food, foods supplements, functional foods, and drinks.

Examples of the food that can be added with the composition for preventing hair loss and growing hairs according to the present invention includes various foods, drinks, a gum, teas, vitamins, functional foods, and the like. Further, the food of the present invention includes special nutrient food (for example, milk formulas, infant/baby food, and the like), processed meat products, fish products, bean curds, starch gels, noodles (for example, instant noodles, all kinds of noodles, and the like), breads, health supplement food, food seasonings (for example, soy sauce, soybean paste, red pepper paste, mixed paste, and the like), sauces, confections (for example, snacks), candy, chocolates, gums, ice creams, milk products (for example, fermented milks, cheeses, and the like), other processed foods, Kimchi, salting food (various kimchis, jangajji, and the like), drinks (for example, fruit beverages, vegetables drinks, soybean milk products, fermented drinks, and the like), and natural seasonings (for example, ramen powder, and the like). However, the present invention is not limited thereto. The above-mentioned foods, drinks, or foods supplements may be prepared by using the typical preparation method.

In addition, the term “functional foods” means food groups having a high value, in which the corresponding food function is applied for a specific object and then expressed by using a physical, biochemical, and biotechnological ways or processed foods that is designed and processed to sufficiently express the body modulating functions related to biological rhythms control, disease prevention, disease recovery, and the like in the body. To be specific, the “functional foods” may be health supplement foods. The functional foods may include cytologically acceptable food supplement additives, and further include a proper carrier, excipient, and diluents that are generally used for preparing a functional food.

In addition, the term “drinks” in the present invention is a generic term for one to quench one's thirst and enjoy the taste, and includes functional drinks. In addition to the addition of the composition for preventing and improving inflammation and immunopathy in a directed ratio as an essential component, the drinks may include other components without specific limitation, and further include various flavoring agents or natural carbohydrates like a general drink.

Furthermore, in addition to the above-mentioned things, the food including the composition for preventing hair loss and growing hairs according to the present invention may include various nutrients, vitamins, minerals (electrolyte), flavoring agents such as synthetic flavoring agents and natural flavoring agents, coloring agents, filler (cheese, chocolate, and the like), pectic acid and a salt thereof, alginic acid and a salt thereof, organic acid, protective colloid viscosity agents, pH regulator, stabilizer, preservatives, glycerin, alcohols, carbonating agents used for carbonated soft drinks, and the like, and may use singly or in combination of the above-mentioned components.

For food including the composition for preventing hair loss and growing hairs according to the present invention, the amount of the composition according to the present invention may be 0.01% by weight to 90% by weight and preferably 0.1% by weight to 40% by weight relative to the total weight of the food. For drinks, the composition may be included in a ratio of 0.001 g to 2 g and preferably 0.01 g to 0.1 g relative to 100 ml, but included in the above range or less in the case of taking for a long period of time for the purpose of health and sanitation or control of health. However, since there is no problem with safety in an effective component, the composition may be used in the above range or more, and thus is not limited to the above range.

Hereinafter, the present invention will be described in more detail with reference to Examples. However, it is obvious by the person skilled in the art that those Examples are only for only illustrating the present invention in more detail, but the range of the present invention is not intended to be limited to those Examples.

### Example 1

### Preparation of Extract of Eucheuma cottonii and Isolation of Hydroxydihydrobovolide Compound

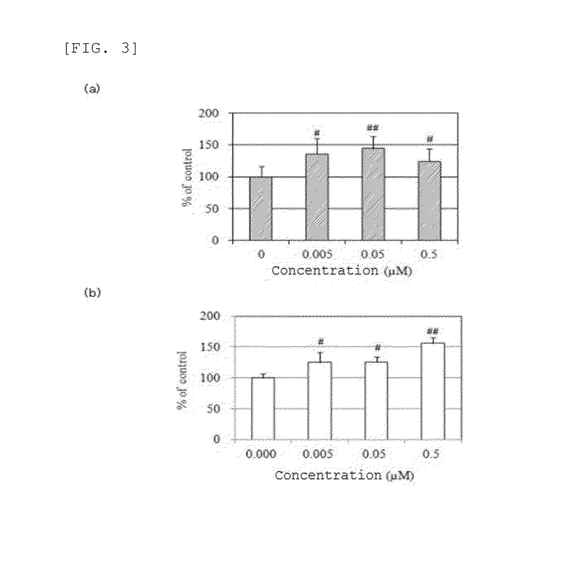

Eucheuma cottonii in a dry state was pulverized, and then extracted and filtered three times by using methanol in 10 times higher than that of a sample weight while stirring. Then, the filtered sample was concentrated by using a rotary vacuum concentrator to prepare a methanol extract in a purity of 99.9%. Then, peaks (see FIG. 2) indicated through a HPLC (YL-9100) were collected by using a reverse phase column (Triart C-18, YMC) under the conditions as listed in the following Table 1, and then concentrated by using the rotary vacuum concentrator.

In order to identify the structures of materials, analyses were performed by using a proton-NMR, a carbon-NMR, and COSY, DEPT, HMBC, and HMQC. As a result, it was confirmed that the compound of the present invention was hydroxydihydrobovolide.

TABLE 1
Conditions for performing HPLC

| | | Flow velocity | | |
|---|---|---|---|---|
| Step | Time (min) | (mL/min) | Methanol (%) | Water (%) |
| 1 | 0 | 1.0 | 60 | 40 |
| 2 | 3 | 1.0 | 60 | 40 |
| 3 | 15 | 1.0 | 80 | 20 |
| 4 | 20 | 1.0 | 100 | 0 |
| 5 | 40 | 1.0 | 100 | 0 |
| 6 | 41 | 1.0 | 60 | 40 |
| 7 | 50 | 1.0 | 60 | 40 |

### Example 2

### Evaluation of Biological Functions of Hydroxydihydrobovolide Compound

In order to confirm whether or not the hydroxydihydrobovolide compound isolated in Example 1 has activity of growing hairs, the following tests were performed, and the cells and samples used for the tests were as follows.

Cell Culture

All of the tests were performed after obtaining approval of Ethics Commission of Medical College, Kyungpook National University. As samples of hairs, hairs in intact state were collected from scalps in the backs of the heads of male hair transplant patients. All of the hair samples were used with the consent of hair transplant patient who provides the samples. The area of subcutaneous fat of scalp including a hair follicle was isolated from the hair sample, and then the hair follicle was isolated by using a binocular microscope. In order to obtain hair papilla cells, the hair papilla was isolated from the hair follicle, and then shifted to a dish that was coated with bovine type I collagen. Then, the hair papilla in the dish was cultured (in an incubator at 37° C. and 5% CO₂) in a DEME (Gibco BRL, Gaithersburg. Md., USA) medium including 20% heat inactivated fetal bovine serum including penicillin (100 U/mL) and streptomycin (100 mg/mL). While the isolated hair papilla was cultured for 10 to 15 days, it was observed that the hair papilla cells were gotten out from the hair papilla. The medium was exchanged once every three days. The hair papilla cells that were grown after getting out from the hair papilla were collected by using 0.25% trypsin/10 mM EDTA, and then passage-cultured by using a DMEM medium including 10% fetal bovine serum. The cells that were passage-cultured twice were used for the test. Hair outer root sheath cells were isolated from the hair sample of hair implant patient by using the same method as the method of isolating the hair papilla cells. The areas of a hair shaft and bulb of hair in a hair follicle were removed to prevent contamination with other cells. The resulting outer root sheath was cultured (in an incubator at 37° C. and 5% CO₂) in a DEME medium including 20% fetal bovine serum. After three days, the medium was exchanged with an epilife medium (Gibco BRL, Gaithersburg, Md., USA) including penicillin (100 U/mL)-streptomycin (100 mg/mL) and fungizone (250 ng/mL). Then, the hair outer root sheath cells were passage-cultured by using the epilife medium. The cells that were passage-cultured three times were used for the test.

<2-1> Measurement of Cell Survival Rate of Hydroxydihydrobovolide Compound

It was investigated whether or not the hydroxydihydrobovolide compound according to the present invention has an effect on cell proliferation in hair follicle cells and cells in the outer root sheath, in which the cells were closely associated with hair growth. To achieve this, the cells were seeded to 96-well plates in 1×10⁴ cells/plate, and then cultured for 24 hours. Then, the samples with various concentrations were prepared by using the medium without FBS and 1% penicillin/streptomycin, and then were treated to the cells. The cells were cultured for 24 hours. After treating with a MTT solution (5 mg/mL) for 3 hours, the MTT was reduced to produce formazan. The medium was carefully removed so as not to remove the formazan, and then 100 μL of dimethyl dulfoxide (DMSO) was added to the cells. After 30 minutes, absorbance of the cells was measured (570 nm).

As a result, it was confirmed that when the hydroxydihydrobovolide compound of the present invention was treated in 0.005 to 0.5 μM, the significant effects on cell proliferation were exhibited as compared with the control group that was not treated with anything. Especially, in the case of 0.05 μM concentration, the cell proliferation was increased by 144% (p<0.05) (see FIG. 3A). In addition, as a result of treating the same test material to the outer root sheath cells, it was confirmed that in the case of the treatment concentrations of 0.005 to 0.5 μM, the cell proliferations were significantly higher than that of the control group, and especially, in the case of 0.5 μM concentration, the cell proliferation was increased by 156%, the highest effect on cell proliferation as compared with the control group (see FIG. 3B).

Accordingly, it was confirmed that the hydroxydihydrobovolide compound of the present invention has effects on increasing cell survival rates in the human hair follicle dermal papilla cells and outer root sheath cells, in which the cells were closely associated with hair growth.

<2-2> Evaluation of Hair Growth using Human Hair Tissue Culture

In order to confirm whether or not the hydroxydihydrobovolide compound of the present invention has an effect on growing hairs, the following test was performed.

The human hair tissues were supplied from an immunology laboratory in Kyungpook National University. The human hair tissues were cultured in an incubator at 37° C. and CO₂ by using Williams E media (Sigma, USA) including 2 mM L-glutamin, 100 U/mL of streptomycin, and 10 ng/ml of hydrocortisone. The test materials with various concentrations were cultured for 6 days, and the lengths of hairs were measured on the third days and sixth day.

It was confirmed from the result of analysis that in the case of the control group that was not treated with anything, the length on the third day was 0.59 mm and the length on the sixth day was 0.93 mm, while when the hydroxydihydrobovolide compound of the present invention was treated in the concentration of 5 μM, the length on the third day was 0.62 mm and the length on the sixth day was 1.38 mm. In other words, it was confirmed that in the case of the sixth day, the length of the hair was increased by at least 1.48 times longer than that of the control group (see FIG. 4).

Accordingly, it is considered that since the hydroxydihydrobovolide compound of the present invention allows the survival rates of human hair follicle dermal papilla cells and hair outer root sheath cells to increase and also has an effect on growing a human hair length, the hydroxydihydrobovolide compound of the present invention is of great value as a pharmaceutical composition or cosmetic composition for improving scalp and preventing hair loss.

As set forth above, according to exemplary embodiments of the invention, since the hydroxydihydrobovolide compound has effects on increasing survival rates of human hair follicle dermal papilla cells and hair outer root sheath cells and also growing a length of hairs, the compound is of great value as a pharmaceutical composition or cosmetic composition for improving scalp or preventing hair loss. Further, since the hydroxydihydrobovolide compound according to the present invention is isolated and purified from Eucheuma cottonii that is red algae and does not induce cytotoxicity, it is stable in the body and thus can be used as a raw material for functional health food.

While the present invention has been shown and described in connection with the exemplary embodiments, it will be apparent to those skilled in the art that modifications and variations can be made without departing from the spirit and scope of the invention as defined by the appended claims.

What is claimed is:

1. A method for preventing hair loss or growing hairs, comprising administering a composition comprising hydroxydihydrobovolide compound represented by the following Chemical Formula 1 as an effective component:

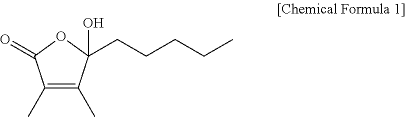

2. The method according to claim 1, wherein the composition further comprises an extract, a concentrate, or dry matter of Eucheuma cottonii.

3. The method according to claim 1, wherein the hydroxydihydrobovolide is included in the composition in a concentration of 0.005 to 10 μM.

4. The method according to claim 1, wherein the compound is manufactured as a formulation selected from the group consisting of creams, gels, patches, spraying agents, ointments, a hardening agent, lotions, liniments, pastas, and cataplamas as a skin external application.

5. The method according to claim 1, wherein the compound is manufactured as any one of the formulations selected from the group consisting of hair tonics, nutritional cosmetic preparations for hair care, scalp treatments, hair treatments, hair rinses, hair shampoos, and hair lotions.

6. A method for increasing survival rates of human hair follicle dermal papilla cells and/or outer root sheath cells comprising administering a hydroxydihydrobovolide compound represented by the following Chemical Formula 1 as an effective component:

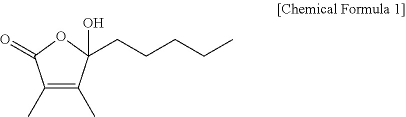

7. A method for lengthening human hair comprising administering a hydroxydihydrobovolide compound represented by the following Chemical Formula 1 as an effective component: [chemical structure]
